(19) United States
(12) Patent Application Publication
(10) Pub. No.: US 2025/0166753 A1
(43) Pub. Date: May 22, 2025

(54) PREDICTIVE HEALTH RISK SCORE TO ENABLE PROACTIVE TRIAGING

(71) Applicants: Regents of the University of Minnesota, Minneapolis, MN (US); Allina Health System, Minneapolis, MN (US)

(72) Inventors: Jiacheng LIU, Minneapolis, MN (US); Jaideep SRIVASTAVA, Minneapolis, MN (US); Lisa KIRKLAND, Minneapolis, MN (US); JoAn HALL, Minneapolis, MN (US)

(73) Assignees: Regents of the University of Minnesota, Minneapolis, MN (US); Allina Health System, Minneapolis, MN (US)

(21) Appl. No.: 18/952,679

(22) Filed: Nov. 19, 2024

Related U.S. Application Data
(60) Provisional application No. 63/600,791, filed on Nov. 20, 2023.

(51) Int. Cl. G16H 10/60; G16H 20/00; G16H 40/20; G16H 50/50; G16H 50/70

(52) U.S. Cl. CPC G16H 10/60; G16H 20/00; G16H 40/20; G16H 50/50; G16H 50/70

(57) ABSTRACT

A system includes a database, a memory storing instructions, and a processor communicatively coupled to the memory and the database. The database stores a dataset comprising previous patient hospital stay data for a plurality of patients. The processor is configured to execute the instructions to generate training data based on the dataset, train a prediction model based on the training data, receive current patient hospital stay data for a current patient, generate a risk score of health deterioration for the current patient based on the prediction model and the current patient hospital stay data, and determine a likelihood of the current patient being transferred to an ICU within a selected period based on the risk score.

### CROSS REFERENCE TO RELATED APPLICATIONS

This application claims priority to U.S. Provisional Patent Application No. 63/600,791, filed Nov. 20, 2023, entitled “KALMAN FILTER BASED FRAMEWORK FOR MONITORING THE PERFORMANCE OF IN-HOSPITAL MORTALITY PREDICTION MODELS OVER TIME” and is incorporated herein by reference.

### BACKGROUND

At the start of a pandemic, such as during the COVID-19 pandemic, hospitals may be overwhelmed with the high number of ill and critically ill patients. There may be a surge in Intensive Care Unit (ICU) demand, which may result in ICU wards running at full capacity, with no signs of the demand falling. As a result, resource management of ICU beds and ventilators may become a bottleneck in providing adequate healthcare to those in need. Accordingly, short-term ICU demand forecasts have become a critical tool for hospital administrators.

In addition, at the beginning of the breakout of a new disease, the healthcare community almost always has little experience in treating patients of this kind. Similarly, due to insufficient patient records at the early stage of a pandemic, it is difficult to train an in-hospital mortality prediction model specific to the new disease. This may be called the “cold start” problem of mortality prediction models.

Further, unlike in a clinical trial, where researchers get to determine the least number of positive and negative samples required, or in a machine learning study where the size and the class distribution of the validation set is static and known, in a real-world scenario, there is little control over the size and distribution of incoming patients. As a result, when measured during different time periods, evaluation metrics like Area under the Receiver Operating Curve (AUROC) and Area Under the Precision-Recall Curve (AUPRC) may not be directly comparable.

In addition, missingness and measurement frequency are two sides of the same coin. A common question is how frequently clinical variables should be measured and laboratory tests be conducted. The answer depends on many factors, such as the stability of patient conditions, diagnostic process, treatment plan, and measurement costs. The utility of measurements varies disease by disease and patient by patient.

For these and other reasons, a need exists for the present invention.

### SUMMARY

Some examples of the present disclosure relate to a system. The system includes a database, a memory storing instructions, and a processor communicatively coupled to the memory and the database. The database stores a dataset comprising previous patient hospital stay data for a plurality of patients. The processor is configured to execute the instructions to generate training data based on the dataset, train a prediction model based on the training data, receive current patient hospital stay data for a current patient, generate a risk score of health deterioration for the current patient based on the prediction model and the current patient hospital stay data, and determine a likelihood of the current patient being transferred to an ICU within a selected period based on the risk score.

Other examples of the present disclosure relate to a system. The system includes a data processor, a prediction model trainer, a prediction analyzer, and a prediction model performance monitor. The data processor is configured to generate training data for a predication model based on previous patient hospital stay data for a plurality of patients and to generate a risk score for health deterioration for a current patient based on the prediction model and current patient hospital stay data. The prediction model trainer is configured to train the prediction model based on the training data. The prediction analyzer is configured to generate an uncertainty score for the risk score and to generate a clinical measurement recommendation for the current patient based on the uncertainty score. The predication model performance monitor is configured to monitor a performance of the prediction model over time.

Yet other examples of the present disclosure relate to a method. The method includes generating a prediction model based on a dataset comprising previous patient hospital stay data including clinical features, vital signs, demographics, and intensive care unit (ICU) status for a plurality of patients. The method includes determining a risk score of health deterioration of a current patient based on the prediction model and current patient hospital stay data to determine a likelihood of the current patient being transferred to an ICU within a selected period. The method includes adjusting treatment of the current patient and/or preparing the ICU to receive the current patient in response to the likelihood of the current patient being transferred to the ICU within the selected period exceeding a threshold.

Examples of the present disclosure generate a model that is predictive, in the sense that the model can generate a probability of what the risk is going to be T time units (e.g., hours or days) in the future. This creates a new capability of proactive triaging based on what the risk is going to be in the future rather than reactive triaging as typically done, in response to a current status of a patient.

### BRIEF DESCRIPTION OF THE DRAWINGS

FIG. 1 is a block diagram illustrating an example system for determining a risk score of health deterioration.

FIG. 2A is a flow diagram illustrating an example process for processing data for use by a model.

FIG. 2B is a flow diagram illustrating an example process for training a model.

FIG. 2C is a flow diagram illustrating an example process for analyzing predictions.

FIG. 2D is a flow diagram illustrating an example process for tracking performance of a model.

FIG. 3 is a block diagram illustrating an example score-based transfer learning method to tackle the cold start problem.

FIG. 4 is a block diagram illustrating example causes of change in predictive model performance over time.

FIG. 5 is a chart illustrating monthly AUROC performance of 2 days ahead in-hospital mortality prediction model for COVID-19 patients.

FIG. 6 is a diagram illustrating an example Kalman filter based framework for estimating model performance over time.

FIG. 7 is a diagram illustrating an example architecture for explaining prediction variance.

FIG. 8 is a diagram illustrating an example variational recurrent model and training loss.

FIGS. 9A and 9B are block diagrams illustrating an example processing system for determining a risk score.

FIGS. 10A-10C are flow diagrams illustrating an example method for determining a risk score.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

As previously described above, at the start of a pandemic, such as during the COVID-19 pandemic, hospitals may be overwhelmed with the high number of ill and critically ill patients. There may be a surge in Intensive Care Unit (ICU) demand, which may result in ICU wards running at full capacity, with no signs of the demand falling. As a result, resource management of ICU beds and ventilators may become a bottleneck in providing adequate healthcare to those in need. Accordingly, short-term ICU demand forecasts have become a critical tool for hospital administrators.

Accordingly, disclosed herein are models, based on existing patient data (such as COVID-19 patient data), to predict if a patient's health will deteriorate below safe thresholds to deem admission into an ICU within a selected period (e.g., within the next 24 to 96 hours). The most important clinical features responsible for the prediction may be identified and narrowed down to the health indicators to focus on, thereby assisting the hospital staff in increasing responsiveness.

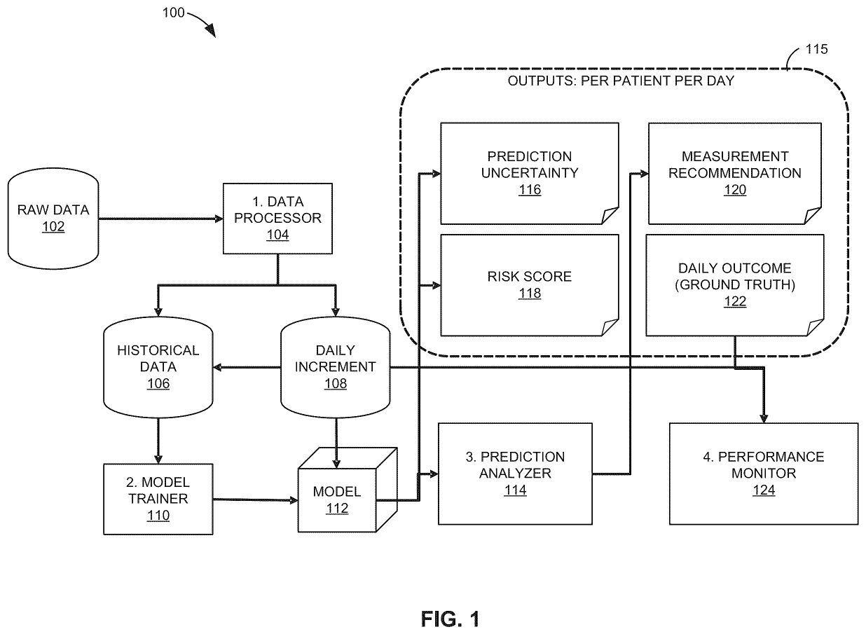

FIG. 1 is a block diagram illustrating an example system 100 for determining a risk score of health deterioration. System 100 include raw data 102, which may include previous patient hospital stay data for a plurality of patients. In some examples, the raw data 102 may include previous patient hospital stay data for a particular hospital and/or for a specific disease or cohort. In some examples, the raw data 102 may include Medical Information Mart for Intensive Care, such as MIMIC3 or MIMIC4 data. The raw data 102 may include deidentified Electronic Healthcare Records (EHR). The raw data 102 may be input to data processor 104.

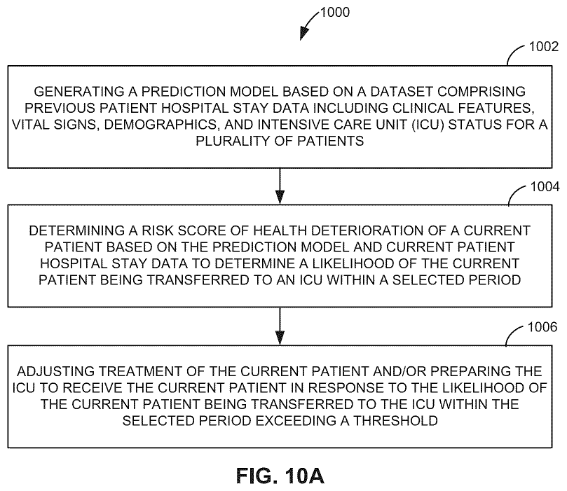

Data processor 104, which is further described below with reference FIGS. 9A and 9B, may receive the EHR records, preprocess the records, and power-transform the data to provide historical data 106. The data processor 104 may then derive features from the historical data and output the training data to model trainer 110. The model trainer 110 may train gradient boosted trees based on the training data to generate model 112 (e.g., a prediction model).

Prediction analyzer 114 may obtain feature importance and identify short-term variables and long-term variables using model 112. For each current patient, the prediction analyzer 114 may compare the feature importance contributions of short-term variables and long-term variables. The ratio may indicate the acuity level of the current patient. The acuity level may provide risk score 118 for the current patient. The prediction analyzer 114 may also generate a prediction uncertainty 116 (e.g., an uncertainty score) indicating the uncertainty of the risk score 118. Based on the risk score 118 and the prediction uncertainty 116, the predication analyzer 114 may also generate a measurement recommendation 120. The measurement recommendation may include, for example, a specific lab test that should be performed, a vital sign that should be measured, etc., that could reduce the prediction uncertainty 116.

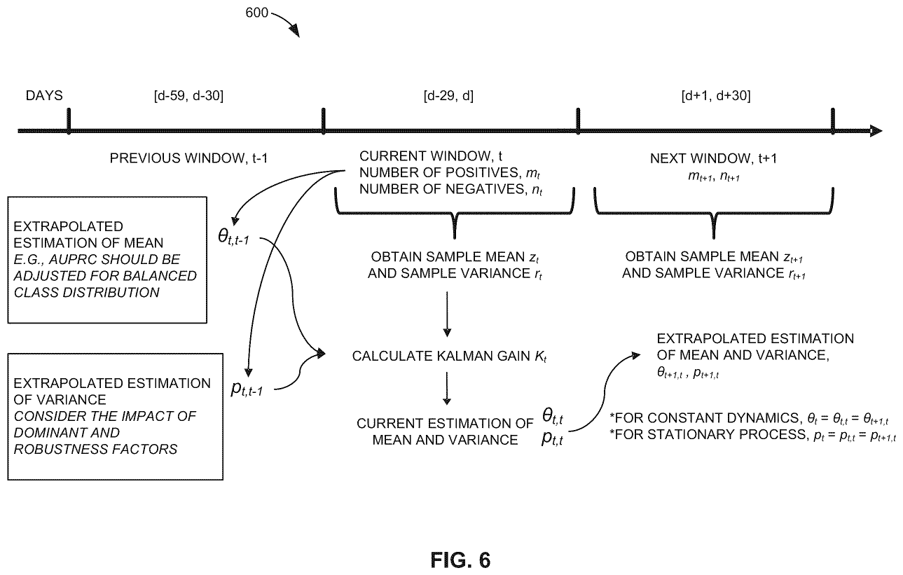

Daily patient outcome 122 is ground truth that may provide a daily increment 108 to update the model 112 and/or historical data 106. Performance monitor 124 may monitor the performance of the model 112 over time as further described below at least with reference to FIG. 4.

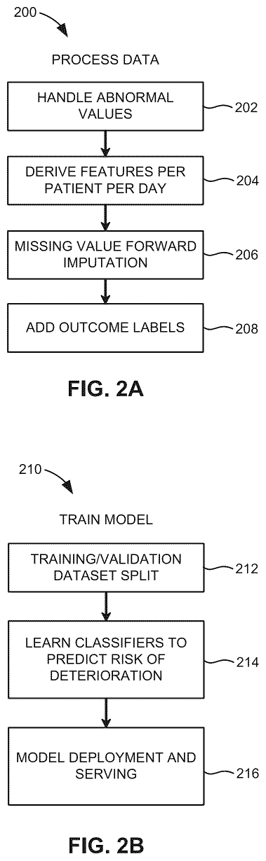

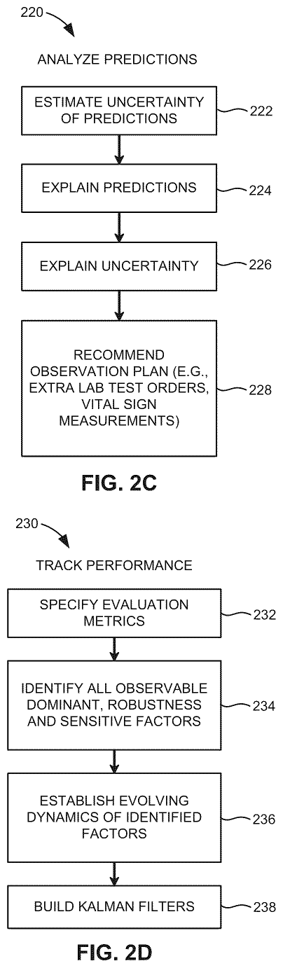

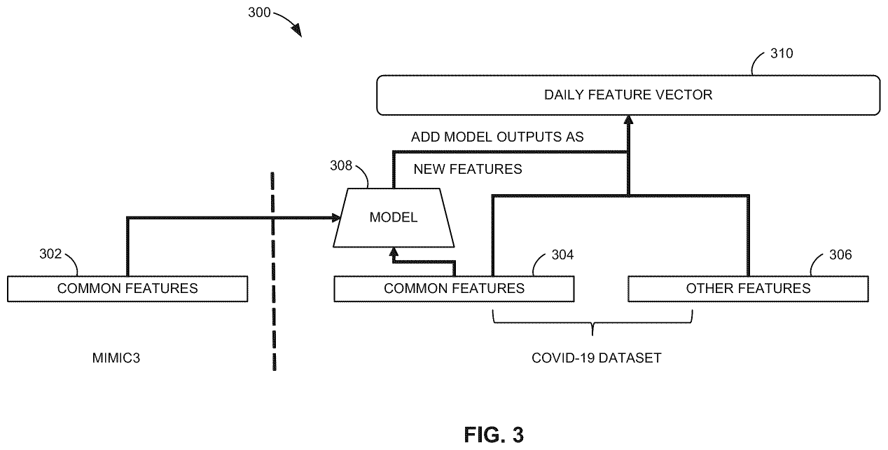

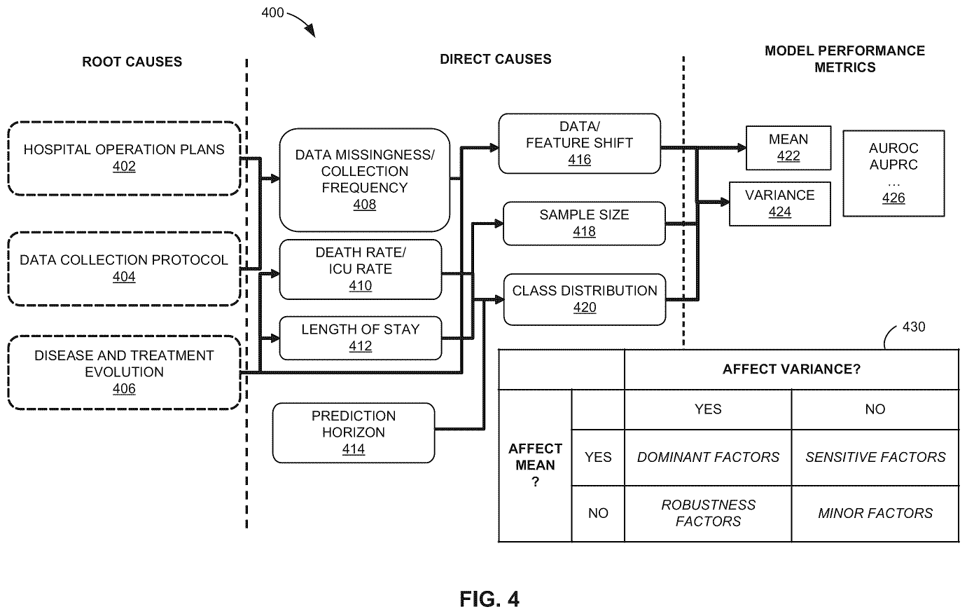

FIG. 2A is a flow diagram illustrating an example process 200 for processing data for use by a model. In some examples, process 200 may be implemented by data processor 104 of FIG. 1 and will be further described below. At 202, process 200 may handle abnormal values. At 204, process 200 may derive features per patient per day. At 206, process 200 may implement missing value forward imputation. At 208, process 200 may add outcome labels 208.

FIG. 2B is a flow diagram illustrating an example process 210 for training a model. In some examples, process 210 may be implemented by model trainer 110 of FIG. 1 and will be further described below. At 212, process 210 may include training/validation dataset split. At 214, process 210 may learn classifiers to predict risk of deterioration. At 216, process 210 may include model deployment and serving.

FIG. 2C is a flow diagram illustrating an example process 220 for analyzing predictions. In some examples, process 220 may be implemented by prediction analyzer 114 of FIG. 1 and will be further described below. At 222, process 220 may estimate uncertainty of predictions. At 224, process 220 may explain predictions. At 226, process 220 may explain uncertainty. At 228, process 220 may recommend observation plan (e.g., extra lab test orders, vital sign measurements, etc.).

FIG. 2D is a flow diagram illustrating an example process 230 for tracking performance of a model. In some examples, process 230 may be implemented by performance monitor 124 of FIG. 1 and will be further described below. At 232, process 230 may specify evaluation metrics. At 234, process 230 may identify all observable dominant, robustness and sensitive factors. At 236, process 230 may establish evolving dynamics of identified factors. At 238, process 230 may build Kalman filters.

The following is an example for processing data, training a model, and generating a risk score as described in association with at least FIGS. 1, 2A, and 2B. In this example, the risk score is used to determine a likelihood of a current patient being transferred to an ICU within a selected period, such as within 24 to 96 hours.

Using a retrospective study with a dataset of 1411 COVID-19 patients from a hospital in the United States of America (USA), it was determined eXtreme Gradient Boosting (XGBoost) performs the best among the models tested when tuning parameters for sensitivity (recall). In some examples, one important feature for the prediction tasks is the maximum respiratory rate, but subsequent features in order of importance vary between models predicting ICU transfer in the next 24 to 48 hours and those predicting for the next 72 to 96 hours.

Medical decompensation may be defined as functional deterioration of a system. Burnout is one of the primary side effects among hospital staff due to a surge in hospital and ICU admissions. Disclosed herein is a combination of the computational capabilities of Machine Learning (ML) algorithms with the interpretability of results to help the hospital staff better plan their limited ICU resources. Hospitalized patients' health condition may improve or worsen during their stay. At times, a patient's health condition is worsened so much that they need to be moved to the ICU. This event, the “Transfer to ICU” may be used as a proxy for health decompensation.

In some examples, the models disclosed herein predict if a patient's health will deteriorate in a selected period, such as in the next 1 to 4 days. The features responsible for the prediction are identified. In some examples, there may be a change in feature importance over the course of disease progression.

The problem may be defined as follows. Given data for a patient p from day d_{-∞} to day di relative to hospital admission, a prediction is made to determine whether the patient will be transferred to the ICU at day dᵢ₊ₓ where i, x∈ [symbol] : x∈[1, 4]. Given the icu_flag(i) for a specific patient at day dᵢ, the outcome variable for change in ICU status in the next x days, icu_change_dₓ(i), where i, x∈ [symbol] : x∈[1, 4] is defined as:

$$icu\_flag ( i ) ⊕ {(}_{icu \_ flag ( i + 1 )} V … {V}_{icu \_ flag ( i + x )} ) \qquad ( 1 )$$

A value of 0 for variable icu_change_dₓ(i) for patient p indicates that p will remain in the general ward, and a value of 1 indicates that they will get transferred to the ICU within the next x days.

The dataset may include data for a plurality of previous hospital patients. The data may include clinical features (e.g., lab results), vital signs, demographics, and an ICU status flag to indicate if the patient was in the ICU on any given day. A dataset based on a vitals daily feature vector may be obtained by grouping data by patient id, date, and vital sign and calculating aggregate values for each group. The aggregate values may include a number of measures, a maximum and minimum value, a mean and standard deviation, and a number of measure two and three standard deviations away from the mean. Linear regression may then be performed on the time series data. Slope and r-squared may be added daily to the feature vector.

Class imbalance may be handled by stratified sampling to divide data into train and test sets, Synthetic Minority Over-sampling Technique (SMOTE), and/or Cost-sensitive learning.

Multiple models were tested, while tuning for hyper-parameters, to select the model with best sensitivity. The model selected is eXtreme Gradient Boosting (XGBoost) that creates boosted decision trees. XGBoost generates ranking of features based on their importance in the decision trees. Two experiments to handle class imbalance were conducted using SMOTE and class weighting. Best performance with class weighting model for predicting ICU transfer in next 2 days, recall: 0.779.

A feature importance score indicates how much the model's performance improved when using a feature's values to split the tree on. The importance metrics in XGBoost are gain, cover, and weight. Each metric results in a slightly different ordering of feature importance. The SHapley Additive exPlanation (SHAP) method may be used to provide robust feature importance. Results of feature importance align with clinical studies that respiratory rate is a vital factor in medical decompensation. Translation of output and feature importance into simple rules is key in assisting the healthcare providers in decision making. For example, a high minimum temperature in a day may indicate transfer to the ICU within the next 4 days. A low minimum blood oxygen in a day may indicate transfer to the ICU within 1 day.

This approach may provide a tool to assist healthcare providers in decision making. The alerts may be tailored such that only if the prediction probability is above a certain threshold, will an alert be issued. A complement to predicting transfers to ICU is predicting transfers from the ICU to the general ward of a hospital or discharge from the hospital. The same data may be used to build another set of models to estimate the likelihood of a person in the ICU getting better within a selected period (e.g., within the next 24 to 96 hours). One caveat to this problem is there may be patients who are discharged from the hospital directly without being moved to the general ward. In those scenarios, it cannot be assumed that the discharge is due to recovery—it could be that patients are moved to hospice for end-of-life care or transferred to a different hospital. This may be addressed by requiring additional labels in the data, such as reasons for discharge or whether patients were moved to hospice. These models could help hospital staff better plan their limited ICU resources by moving those on a path to recovery out of the ICU while bringing those whose health is likely to deteriorate into the ICU.

The following example relates to the cold start problem for building a predication model when insufficient data is available. This example also involves processing data, training a model, and generating a risk score as described in association with at least FIGS. 1, 2A, and 2B. In this example, the risk score is used to determine a likelihood of a current patient dying within a selected period, such as within 3 days.

As previously described above, at the beginning of the breakout of a new disease, the healthcare community almost always has little experience in treating patients of this kind. Similarly, due to insufficient patient records at the early stage of a pandemic, it is difficult to train an in-hospital mortality prediction model specific to the new disease. This may be called the “cold start” problem of mortality prediction models.

Accordingly, disclosed herein the cold start problem of 3-days ahead mortality prediction models are addressed by the following two steps: (i) Train XGBoost and logistic regression (e.g., 3-days ahead) mortality prediction models on a patient dataset (e.g., MIMIC3, a publicly available ICU patient dataset); (ii) Apply those prediction models to patients and then use the prediction scores as a new feature to train (e.g., 3-days ahead) mortality prediction models. Retrospective experiments were conducted on a real-world COVID-19 patient dataset (n=1,287) collected in the United States from June 2020 to February 2021 with a mixed cohort of both ICU and Non-ICU patients. Since the dataset is imbalanced (death rate=7.8%), the focus is primarily on the relative improvement of Area Under the Precision-Recall Curve (AUPRC). Models were trained with and without MIMIC3 scores on the first 200, 400, . . . , 1000 patients respectively and then tested on the next 200 incoming patients. The results showed a diminishing positive transfer effect of AUPRC from 5.36% for the first 200 patients (death rate=5.5%) to 3.58% for all 1,287 patients. Meanwhile, the Area Under the Receiver Operating Curve (AUROC) scores largely remain unchanged, regardless of the number of patients in the training set. What's more, the p-value of t-test suggests that the cold start problem disappears for a dataset larger than 600 COVID-19 patients. In summary, the cold start problem is mitigated via the method disclosed herein.

The COVID-19 pandemic has drawn huge attention from researchers to study its biological traits, develop new vaccines and treatments, guide public health policies, build prediction models, and search for answers of perhaps the most important question, what lessons can be learned from this pandemic? In this disclosure, from a health informatics point of view, it is disclosed how one can do better at the early stage of an outbreak of a previously unknown disease. Specifically, the difficulties of training in-hospital mortality prediction models in the early days of COVID-19 is examined. To do that, COVID-19 patient electronic healthcare records (EHR) data was used as extracted from Abbott Northwestern Hospital at Minneapolis, MN, between Jun. 1, 2020 to Feb. 28, 2021, roughly the period right before extensive vaccination roll out and the prevalence of the COVID virus delta variant.

The phrase “cold start” has its origins in the domain of automotive, referring to the starting of a vehicle engine at a lower temperature relative to its operating temperature. Just as engines have lower limits of operating temperatures, at least a certain amount of data is required to effectively train predictive models. Due to insufficient patient records at the early stage of a pandemic, it is difficult to train an in-hospital mortality prediction model specific to the new disease. Thus, this is named the “cold start” problem of mortality prediction models.

Transfer learning is a natural approach to the cold start problem. In fact, this is also what human physicians were doing at the beginning of the pandemic, that is, leveraging biomedical knowledge and experience of treating other diseases. Besides, studying the cold start problem via transfer learning approaches may also bring insights about the new disease. If the positive transfer effect persists, as the new disease dataset continues to grow bigger, this may suggest connections between already known diseases and the new one. Otherwise, if the positive transfer effect drops to zero at some point, this may signal the end of cold start. There are many issues preventing these prediction models from being used in the production environment, such as potential data bias in the training data, the heterogeneity of cohorts, data interoperability problems and nuances in clinical variables collection practice in each hospital. Therefore, sometimes it may be desirable for every hospital to train their own prediction models. Studying the cold start problem may indicate how many data records are required to train a predictive model.

In an ideal setting, given two datasets, one can learn from the source dataset (e.g., MIMIC3 dataset), and then apply models to the target dataset (e.g., COVID-19 cohort), as long as two datasets share the same feature space. This is called domain adaptation, which is a subarea of transfer learning. However, in reality it is more complicated. First of all, since in the early stage of a new disease, the practice guide of what laboratory tests to order may be subject to change as more knowledge is gained and hence the common feature space between source and target domains will also change accordingly. Secondly, due to the sparseness and irregular measurement problem, some common features are basically not helpful at all. Therefore, transfer models may be trained on vital signs, namely heart rate, respiration rate, blood pressure, saturation of oxygen, and temperature, which are almost guaranteed to be common features across datasets and even hospital systems.

Machine learning techniques have been applied to aid triaging decisions for COVID-19 patients and have shown promising results. Herein, the focus is mainly on results based on Electronic Health Records (EHR) data rather than chest CT images. Some previous approaches build logistic regression models to predict whether a patient will develop critical conditions such as admission to ICU, need of invasive ventilators, or death. These models are based on clinical symptoms, lab results, radiology reports, medical history, and demographics. Other previous approaches report an AUROC of 0.88 on independent test sets. Other previous approaches validate their model on 5 cohorts collected across hospitals in Belgium, China, and Italy. The AUROC ranges from 0.84 to 0.89. However, these models are one-shot classification models working at the time of admission, rather than models making daily predictions. A publicly available COVID-19 electronic medical records dataset was released consisting of 485 COVID-19 patients which can be used for various research purposes. Herein, this dataset is referred to as the Wuhan Dataset in the rest of this disclosure, as all patient data is collected from Wuhan, China. In others work, XGBoost models are built to predict risk of mortality for patients' last day records. The result shows 97% accuracy on test and over 0.9 F1 score for both who survived and who died.

Learning with few samples is the active research area of few shots learning, transfer learning or domain adaptation, and sometimes semi-supervised learning. Since few shots learning usually requires embeddings of external prior knowledge, disclosed herein are transfer learning or domain adaptation techniques. While lots of studies use medical images, there are relatively few studies that use EHR data. Among them, variational recurrent adversarial deep domain adaptation combines the idea of domain adversarial training and variational recurrent neural networks to learn domain invariant hidden features and achieved decent improvement for minority race groups in the MIMIC3 dataset. However, as far as known, there is no literature directly studying the cold start problem of in-hospital mortality prediction models (e.g., for COVID-19) based on EHR data as disclosed herein. Accordingly, disclosed herein is a transfer learning method for risk of mortality prediction models based on EHR data (e.g., for COVID-19). The positive transfer effect was verified via retrospective experiments.

As the size of the COVID-19 dataset grows, a diminishing positive transfer effect is observed. The results suggest that the cold start problem ends at a size equal to about 600 patients. In other words, assuming the same death rate around 8%, any researcher who wants to train their own “3-days ahead in-hospital mortality” prediction models without suffering the cold start problem should have a dataset of at least 600 COVID-19 patients.

Further described below are the patient selection criteria, summary statistics, and data processing steps. Some real-world data restrictions are also described. Next, problem definitions are introduced and the proposed transfer learning method is described. The impact of cold start is first estimated by analyzing the feature importance. Then, head to head random split experiments were used to compare 3 methods: (i) the baseline model is trained solely on COVID-19 data; (ii) adding prediction scores made by the model trained on all phenotypes of MIMIC3 patients; (iii) adding prediction scores made by the model trained on selected phenotypes of MIMIC3 patients. Further, to illustrate the decreasing positive transfer effects intuitively, models were trained on the first 200 to 1000 patients with a rolling window size of 200 patients.

Adult COVID-19 patients admitted to Abbott Northwestern Hospital at Minneapolis, MN, between Jun. 1, 2020 to Feb. 28, 2021 were included in the study. The data collection plan was approved by the Institutional Review Board (IRB). The following inclusion and exclusion criteria were adopted when extracting EHR data from the database.

### Inclusion Criteria:

1) Age>=18.
2) Diagnosed with COVID-19.
3) Agree Data Usage for Research==Yes.
4) Hospitalized either in general wards or ICU, with a known end of stay outcome (died or survived).

### Exclusion Criteria:

1) Age<18.
2) Agree Data Usage for Research==No.
3) Only has records of emergency visits.
4) Patients who had not been discharged yet when data was collected.

Hospital stays less than 3 days or with 9 or less measurements during the entire stay were removed due to the concern of insufficient data to make predictions. Every patient has only one stay in the extracted dataset. The above criteria resulted in a dataset of 1,287 patients and a death rate of 7.77%, n=100. This dataset will be referred as Allina COVID-19 Dataset in the rest of this disclosure. It is noted that there is a mixed population of both ICU and Non-ICU patients. During one stay, a patient may have multiple ICU admissions.

While many previous publications suggest that Lactate Dehydrogenase (LDH), D-dimer and Ferritin are effective predictors of COVID-19 end-of-stay mortality, unfortunately in the collected data, they are barely usable because of data sparseness. On average, there are less than two valid values of the following key lab results per patient stay. Given that the average length of stay is around 12 days, lab results are very sparse. Though not shown here, it was empirically verified that adding these laboratory results does not improve but even slightly undermines the performance of predictive models, mainly because most of the data records are imputed values.

Static demographic variables and regularly collected vital signs were used. Sequential Organ Failure Assessment (SOFA) scores were also collected.

To begin with, abnormal values, such as SpO₂>100 and negative blood pressures are discarded. To tackle sparseness and irregular measurement intervals of vital signs, it was decided to aggregate all vitals measured on the same day into one daily vital vector. Statistical features like mean, min, max, slope, and r² are derived for each vital sign, where r² is the coefficient of determination. Moreover, a binary imputation mask variable is added for every vital, indicating whether the value on that day is imputed or not. There are 7 vital signs. In the end, the sum of number of vital measurements on any given day is added as a global feature. Therefore, a daily vital vector is of size (5+1)×7+1=43.

Then, one-hot encoders are used to transform categorical variables like race and ethnicity. American Indians or Alaska Native, Hawaiian or Pacific Islander and Patient Declined to Answer are grouped into one race group so that there are 4 race groups and 3 ethnicity groups. Missing static variables with population median are imputed. Bringing on gender and age, there are 9 static variables. Together with the days since admission, 10 variables are appended to the daily vital vector.

Additionally, there are daily ICU flags denoting if the patient is in the ICU. In total, a daily feature vector of dimension 43+10+1=54 is constructed for every patient every day. Upon missing values, forward imputation is applied, that is to use the most recent value as the default value. If the variable is missing on the first day, imputed values will be conditioned on the ICU status on that patient day.

Medical Information Mart for Intensive Care, or MIMIC-III in short, is a large, open-source, deidentified database of about 40,000 ICU patients. It contains clinical notes, ECG data, time series of vital signs, laboratory test results and assessment scores. Herein, the hourly level MIMIC3 data are aggregated to daily feature vectors following the same processing steps mentioned above. Since MIMIC3 is deidentified, vitals are the only set of common features shared between MIMIC3 and Allina COVID-19 Dataset. What's more, patients in MIMIC3 are classified into 25 nonexclusive phenotypes.

The three days ahead (or two days ahead, or four days ahead, etc.) in-hospital mortality prediction problem is now described. After data processing, the time granularity is in days. Two extreme choices would be 1 day ahead risk of mortality prediction and the end of stay mortality prediction. One day ahead prediction suffers the most from imbalanced labels and it leaves little intervention time for physicians. While the end-of-stay prediction task aims to characterize the hazard function during the entire stay, it provides little clues to the imminence of patient deterioration. In the worst case of limited resources or short of staff, physicians would likely take care of rapidly deteriorating patients. Thus, the prediction score should somehow reflect the probability that which patient will be deteriorating in the near future. Considering the trade-off above, it was selected to predict the risk of mortality in 3 days (but the model is also applicable to 2 days, 4 days, etc.). According to the data selection criteria mentioned above, all patient stays are at least 3 days long.

Given the event of interest to be death, formally, assume the survival function S(t) where is the day since admission, the binary classifier is effectively estimating S(t+3)-S(t) conditioned on all covariate history available up to the current time t. Since in the hospital production environment, new data are loaded into the data warehouse every morning, time t denotes the start of the day. This also means that there is a one day delay in terms of data availability. In fact, x₁, x₂ . . . , xₜ₋₁ data is used which are available in the morning of day t to predict if the patient is going to die in day t, t+1 or t+2. This difference of time subscriptions is not made in later discussions. Whenever x₁ . . . t is mentioned, it just means all the covariate history available at time t. Though bear it in mind that x_{1 . . . t}=x₁, x₂ . . . , xₜ₋₁. As disclosed herein, the model may produce daily predictions for every patient with new incoming data.

Computer-aided clinical decision support systems may be deployed in many hospitals, contributing to daily routine triaging and risk prediction tasks. When facing the early stage of an outbreak of an unknown pandemic, the performance of previous models and systems is likely to drop dramatically. Therefore, it is necessary to train new models specific to the new threats. However, given a death rate of around 10%, it is difficult to train and tune hyper-parameters by k-fold cross-validations, since there are only few positive samples, let alone deep learning EHR models which requires many more records.

In such cases, the primary goal is to reduce cold start effects, namely to boost the performance of predictive models by selectively using previous data, features, or models. Traditionally, this can be done by either zero-shot/few shots learning or transfer learning. The secondary goal is to study the potential connections between the new disease and existing knowledge of deterioration paths.

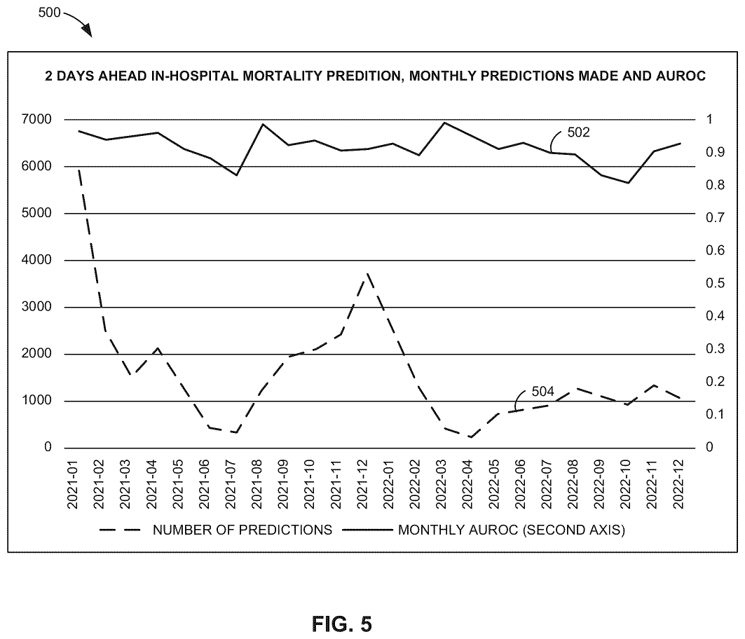

Accordingly, FIG. 3 is a block diagram illustrating a score-based transfer learning method 300 comprising three steps to tackle the cold start problem. Due to the small size of initial data sets, deep models and deep transfer learning methods are not applicable.

Given the source dataset Dₛᵣₑ and the target dataset D_{tgt}:
1) Train a classifier fₜᵣₐₙₛ on shared features of D_{src}.
2) Add fₜᵣₐₙₛ(D_{tgt}) to D_{tgt} as a new feature column. The augmented dataset is called D′_{tgt}.
3) Train a classifier on D′_{tgt}, using all the features.

This process is illustrated by FIG. 3. COVID-19 is used as an illustrative example. First, given a small number of COVID-19 EHR, the MIMIC3 data and a prediction target, a classifier is trained on the common features 302 and 304 shared by both datasets (while excluding other features 306). Let us call this classifier “MIMIC3 model”. As mentioned above, MIMIC3 data are aggregated into daily feature vectors 310. The common daily features are systolic/diastolic blood pressure, heart rate, respiration rate, temperature, SpO₂ and statistical features derived from raw data. Then, this model 308 is applied to COVID-19 data records, of course, with only common features as inputs. Prediction scores produced by the MIMIC3 model are added as additional feature columns for COVID-19 patients. In the last step, a classification model is trained on the augmented COVID-19 dataset. The hope is that the COVID model itself can select important features and determine when to or not to use the MIMIC3 score, conditioned on the patients' covariates. And finally, with enough COVID-19 data, the COVID-19 risk of mortality prediction model will gradually adapt to the COVID-19 population.

Positive transfer effects, namely the performance improvement after adding MIMIC3 scores into the feature set, may imply similarities between the new disease and deterioration paths. If there were no positive transfer effects at all, then it suggests either defined classes cannot be easily separated in the common feature space, or the new disease may be completely different from the selected source domain.

Retrospectively, to measure the impact of cold start, the early data may be left out, all other data may be used to train a model, then tested on the early data, which may be a good indicator of the upper bound of model performance at early times. However, a pragmatic measurement may be taken of the cold start effect. That is to say, how much can the model performance be improved? Therefore, the positive transfer effect drops to zero at some point, as more and more new disease data become available, which may signal the end of cold start. The benefit is that this approach is not retrospective. There is no need to wait for a large amount of data.

In experiments, the following three methods were tested and compared.
1) M1 Baseline Method: Use only COVID-19 data to train predictive models. No scores from transfer models.
2) M2 Transfer from all phenotypes in MIMIC3: All processed MIMIC3 data are used as source domain D_{src}. Common features of MIMIC3 and Allina COVID-19 Dataset are discussed above. There are 25 phenotypes in MIMIC3. Notably in MIMIC3, phenotype labels are non-exclusive. One patient may be categorized into multiple phenotypes at the same time. The data process resulted in 14,666 patients, 351,830 ICU days and a death rate of 11.97%, (n=1,756).
3) M₃ Transfer from selected phenotypes in MIMIC3: Due to the nature of the COVID disease, pulmonary disease phenotypes are selected as listed below as the source domain D_{src} to train classifiers.
   Chronic obstructive pulmonary disease chronic
   Other lower respiratory disease acute
   Other upper respiratory disease acute
   Pleurisy; pneumothorax; pulmonary collapse acute
   Pneumonia acute
   Respiratory failure; insufficiency; arrest acute
   Septicemia (except in labor) acute
   Shock acute
      The process of selected MIMIC3 data resulted in 8,983 patients, 215,535 ICU days and a death rate of 16.04%, (n=1,441).

Methods M1, M2 and M3 were tested in a head-to-head comparison. Then simulation experiments were run to demonstrate the cold start and positive transfer effects during the data collection period, June 2020 to February 2021.

For the experiments, XGBoost models were trained for all 3 candidate methods. Hyper-parameter tuning of max_depth, learning_rate, gamma (early stop), reg_lambda (L2 regularization) were done by 5-fold cross validation for the highest AUPRC, using the training data given in each experiment settings. The reg_alpha=0 (L1 regularization) and objective= “binary: logistic” were fixed. All data processing, model training and evaluation tasks were implemented in python 3.9.7. All experiments were running on CPUs.

To evaluate the model performance on imbalanced datasets, the Area Under Precision-Recall Curve (AUPRC) and stress less on Area Under Receiver Operating Curve (AUROC) were evaluated. Evaluation functions are built-in functions in scikit-learn. AUPRC tells one how expensive the trade-off is the when tuning the decision thresholds. A higher AUPRC score is considered better.

In order to reduce the variation of results in the 3-days ahead mortality prediction task, not only the percentage of positive labels in train/validation/test split are controlled, but also roughly control the lengths of stay distribution. Since the scikit-learn package has implementations of Group k-fold Split and Stratified k-fold Split, but not Group Stratified k-fold Split, a function (Algorithm 1) is implemented herein for the use of splitting data into k-fold by groups and labels. Data records from one patient can not appear in the train and test split at the same time.

Algorithm 1: GroupStratifiedKfold

```
Input data, group_name, label
Parameters n, k, [symbol]   equal cut the length distribution into n bins.
partition the data into k-fold
Output a list of kfold indices.
for i in 1 to k do
  kfold[i] ← { } [symbol]   Initialize each part with an empty set
end for
pa_pos ← data[label = 1].patientid
pa_neg ← data[label = 0].patientid
pa_pos_bins = splitbyLengthsOfStay(pa_pos) [symbol]   results in n exclusive parts
pa_neg_bins = splitbyLengthsOfStay(pa_pos)
for i in 1 to n do
  temporary_pos ← randomKSplit(pa_pos_bins)
  temporary_neg ← randomKSplit(pa_neg_bins)
    for j in 1 to k do
  kfold[j] ← kfold[j] ∪ temporary_pos[j] ∪ temporary_neg[j]
  end for
end for
return kfold
```

First, deidentified patient IDs are split by their binary end-of-stay outcome labels. Then take all survivors as an example, survivor patient IDs are further split into n bins by their total length of stay. In each of the bins, the data is randomly partitioned into k folds. These steps are repeated for the other class of patients, then the resulting data is combined to form the k parts of group stratified patient IDs with their length distribution controlled.

In the experiments, the model was updated and retrained on all available data up to the last day of August 2020, September 2020, . . . , January 2021. June 2020, July 2020 and February 2021 were not considered because of insufficient data for the baseline method M1. To begin, all the data up to Aug. 31, 2020 was considered. Then, an 80/20 group stratified split was used for the train/test data respectively. All three methods were trained on the same training set and tested on the same test split. AUPRC and AUROC were recorded as performance metrics. This “random group stratified split-model training-model testing” loop was repeated 100 times for every end date listed above.

There is a relative AUPRC improvement as time goes by and more data become available. Referencing the cumulative number of patients, the estimated zero positive transfer effect size is about 600 patients with a death rate around 7%. Therefore, the estimated size where cold start effects completely vanish is also 600 patients.

To show the cold start effect and positive transfer effect in real world COVID-19 data, a 200 patients rolling test scheme is adopted. That is, without any random split, patients are ordered chronologically by their admission dates. The first training set includes the first 200 admitted patients, the first test set will be the next 200 patients. So on and so forth, from 200 to 1000, with a step size of 200, the model is retrained on all training data and the model was tested on the next 200 admitted patients.

During the experiments, it was found that random stratified train/test split without controlling the length of stay (LOS) distributions cause large variations in the results. That is because the models make a prediction at every time step and only the last three days can be positive. The level of “imbalanceness” could be large if LOS distributions are left uncontrolled. Thus, algorithm 1 is implemented. The number of bins is a parameter that controls the granularity of similarity between the LOS distribution of training set and the LOS distribution of test set. The bigger the n is, the less variation from LOS differences.

The advantages of the proposed methods include: (i) It is a lightweight transfer learning architecture which requires no assumption on the learning algorithms, as long as they can produce prediction scores. For example, a deep learning model may be trained on an abundant source of data domain D_{src}, then that model may be applied to the much smaller D_{tgt} since it would be hard to tune deep models on small datasets directly. (ii) The proposed method allows the flexibility that multiple models may be trained on D_{src} and all of them may be added to the target dataset. (iii) The proposed method will not significantly increase the training time of classifier on D_{tgt} because it is adding only a few feature columns.

In some examples, the common feature space of vital signs may not be powerful enough to capture the difference between dead and survivors of COVID-19. Vital signs were used in this disclosed method due to the limitation of Allina COVID-19 Dataset. This could be a double-edged sword in a sense that on one hand, the model may be applicable to a wide range of diseases since almost every EHR dataset includes vitals. On the other hand, not including lab test results may significantly undermine the capability or predictive models. In addition, MIMIC3 is an ICU database while a mixed population of ICU and general ward patients exists. Further, baselines using other non-deep transfer learning techniques were not set up.

To tackle the cold start problem of training predictive models, a score-based transfer learning method has been disclosed herein. The disclosed method demonstrates a 5.36% improvement of AUPR when only 200 COVID-19 patients' data are available. Given the features derived as described above, experiments suggest that 600 is a decent size of COVID-19 EHR training set to predict risk of in-hospital mortality 3 days ahead.

The following example relates to monitoring the performance of a prediction model over time. This example involves performance monitor 124 of FIG. 1 and the tracking performance process 230 of FIG. 2D. In this example, a Kalman filter based framework is used to monitor the performance of in-hospital mortality prediction models over time.

Unlike in a clinical trial, where researchers get to determine the least number of positive and negative samples required, or in a machine learning study where the size and the class distribution of the validation set is static and known, in a real-world scenario, there is little control over the size and distribution of incoming patients. As a result, when measured during different time periods, evaluation metrics like Area under the Receiver Operating Curve (AUROC) and Area Under the Precision-Recall Curve (AUPRC) may not be directly comparable.

Accordingly, as disclosed herein, for binary classifiers running in a long time period, these performance metrics are adjusted for sample size and class distribution, so that a fair comparison can be made between two time periods. Note that the number of samples and the class distribution, namely the ratio of positive samples, are two robustness factors which affect the variance of AUROC. To better estimate the mean of performance metrics and understand the change of performance over time, a Kalman filter based framework is used with extrapolated variance adjusted for the total number of samples and the number of positive samples during different time periods. The efficacy of this method was demonstrated first on a synthetic dataset and then retrospectively applied to a 2-days ahead in-hospital mortality prediction model for COVID-19 patients during 2021 and 2022. Further, the disclosed prediction model is not significantly affected by the evolution of the disease, improved treatments and changes in-hospital operational plans.

Area under the Receiver Operating Curve (AUROC) is widely used as an evaluation metric of predictive models with binary outcomes. In health informatics, such prediction targets can be diagnosis of a particular disease, malignancy of a tumor, risk of ICU transfers and risk of in-hospital mortality. A typical research workflow involves derivation and training of the predictive model, then the model will be evaluated on a held-out test dataset. Unless the model is deployed, no further performance metrics (such as AUROC) will be recorded.

Continuous monitoring is essential for any predictive model to be operationalized, so that adjustments of model parameters (such as decision thresholds), and decisions of whether the model is outdated can be properly and timely made. Unlike in a controlled environment where the performance is either evaluated on desired class distribution and sample size (i.e., in a clinical trial), or reported only once (i.e., in a machine learning study), tracking model performance over time requires multiple and regular tests of the model. This brings challenges in an evolving environment because the size and the class distribution (namely the ratio of positive class, if binary prediction target assumed) of the incoming data batch is no longer the same as of the initial training/validation dataset. To be more specific, the number of samples and the class distribution are two robustness factors which affects the variance of performance metrics like AUROC. Besides, a robustness factor for one performance evaluation metric can be a dominant factor of another evaluation metric. For example, number of ground truth positive samples only affects the variance of AUROC, but both mean and variance of Area under the Precision-Recall Curve (AUPRC).

Herein, the focus is on the problem of tracking the mean AUROC of binary classifiers over time in an environment of changing sample size and positive ratio. Since small and imbalanced datasets are quite common in the healthcare domain, a bootstrapping method will not always work. Therefore, a one dimensional Kalman filter based framework is disclosed, where a simple constant dynamic is employed, and the variance of the next time step is extrapolated in a sample size/positive ratio adjusted way. Furthermore, upon the appearance of extremely skewed class distribution, e.g., only 10 positive cases and 490 negative cases, variance upper bound is used, which is adjusted for the sample size and positive ratio, instead of the sample variance inferred from the current test data batch. The number of positive and negative cases must be taken into account as dominant, robustness or sensitive factors of performance change, as they may swing a lot in a real world scenario. The Kalman based framework is flexible enough to incorporate with different evaluation metrics under different assumptions of sample size and class distributions.

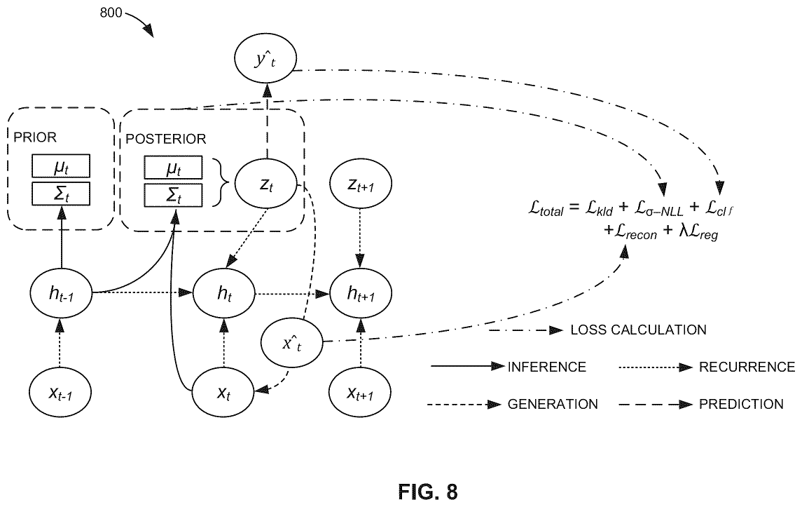

The disclosure can be summarized as follows.
A layered model for performance change analysis. It is pointed out that the number of positive and negative samples must be considered when explaining the change of model performance, as illustrated in FIG. 4.
A Kalman Filter based framework for estimating model performance over time. Following the analyses of dominant factors, sensitive factors, and robustness factors, a Kalman filter is a natural choice when the combined effects of these factors weigh in (FIG. 6). Further, rationales to use the variance upper bound instead of the sample variance are provided, if the number of positive cases is extremely low.
Retrospective filtered performance of 2 days ahead in-hospital mortality prediction model for COVID-19 patients. The disclosed algorithm was applied to a set of COVID-19 patients admitted between 2020 June and 2022 December. The model was trained on 2020 data. Then the test performance on the year of 2021 and 2022 is reported. The result suggests a consistently high prediction performance.

FIG. 4 is a block diagram illustrating example causes 400 of change in predictive model performance over time. As shown in FIG. 4, root causes may include hospital operation plans 402, data collection protocol 404, and disease and treatment evolution 406. Direct causes may include data missingness/collection frequency 408, death rate/ICU rate 410, length of stay 412, and prediction horizon 414. Direct causes may also include data/feature shift 416, sample size 418, and class distribution 420. The model performance metrics include mean 422 and variance 424 for AUROC and/or AUPR 426. As shown in table 430 in FIG. 4, mean is affected by dominant factors and sensitive factors and not affected by robustness factors and minor factors. Variance is affected by dominate factors and robustness factors and not affected by sensitive factors and minor factors.

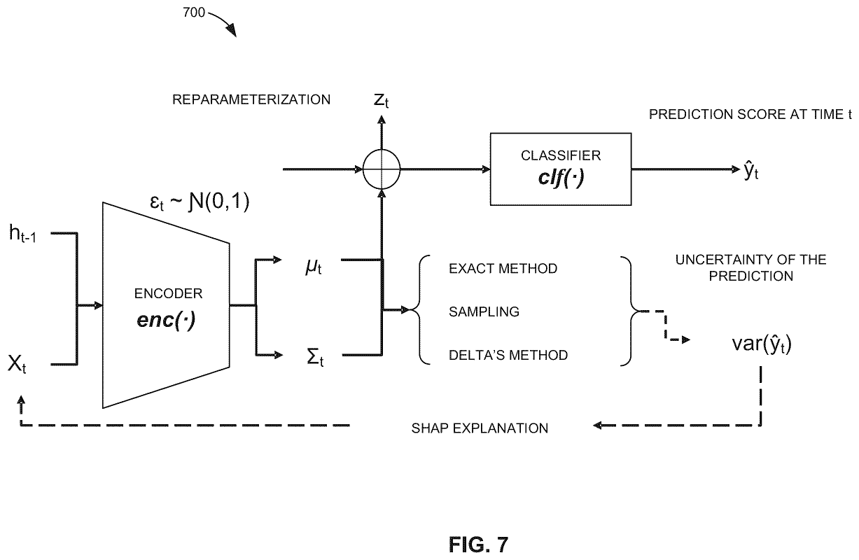

The direct motivation for this disclosure is demonstrated in chart 500 of FIG. 5, where the line 502 is AUROC, calculated monthly for a 2 days ahead COVID-19 in-hospital mortality binary classifier during 2021 and 2022. The prediction model scores the hospitalized COVID-19 patients daily, indicating the risk of mortality in the next two days. The prediction model is trained only based on data in year 2020, and has not been retrained afterwards. The line 504 is the total number of predictions made in that month. These are COVID-19 patients admitted to general wards or Intensive Care Unit (ICU) of Abbott Northwestern Hospital at Minneapolis from 2020 to 2022. The number of predictions shows an obvious seasonal trend as expected, as there are fewer patients in Spring and Summer.

It is worth noting that the valleys of line 504 correspond to the period when big fluctuations happens in the line 502. Recall the analysis made in FIG. 4, this is likely no coincidence. Given the fact that the 2 days ahead COVID-19 in-hospital mortality binary classifier is trained solely on 2020 data, it was evaluated whether, if any of the changes in disease variants, treatment approaches, vaccination status, has an impact on the performance of the model. These questions will be answered by the disclosed method as described below. Also notice that, the number of predictions made in each month is affected by the number of incoming patients and the average length of hospital stay at the same time, e.g., same number of patients with shorter length of stays, there will be a smaller number of predictions per month in total.

Two days ahead in-hospital mortality prediction for COVID-19 patients models are trained to predict risk of in-hospital mortality in the next 2 days for both non-ICU (referred as “floor” patients) and ICU patients. The prediction horizon is determined to be 2 days since 1 day ahead prediction suffers the most from imbalanced labels and it leaves little intervention time for physicians. While the end-of-stay prediction task aims to characterize the overall risk during the entire stay, it provides little clues to the imminent danger of deterioration. According to the data selection criteria mentioned herein, all patient stays are at least 2 days long.

If a patient is transferred to the ICU at least once during the entire hospital stay, it will count as a patient in the ICU stratum. In terms of end-of-stay outcomes and lengths of stay, while there is a clear distinction between the floor patients and ICU patients, the annual change is minimal. The dataset includes 7,080 patients from 2020 to 2022 with an average death rate of 10.95% and ICU rate of 27.2%.

Area Under the Receiver Operator Curve (AUROC) is a common evaluation metric in biostatistics and machine learning studies. Following the Mann Whitney estimator of AUROC 4.2, there are two main methods for estimating the variance of AUROC, Obuchowski's method and DeLong's method. Bootstrapping is another commonly used empirical estimator of variance. However, in the case of few samples available, the performance of which quickly deteriorates. Kalman filters are widely applied to various areas.

Lots of research efforts have been devoted to triage or predicting risk of in-hospital mortality for COVID-19 patients during the last few years. In the area of predictive modeling, there are studies based on time-varying variables and images. The algorithms range from non-deep methods like XGBoost to neural networks, with problem formulations of both supervised and transfer learning.

Upper bound of the sample variance of AUROC may be derived from DeLong's method for estimating the variance of AUROC. This upper bound is used later in case there is very few positive samples in an observation window as a conservative estimation of variance of AUROC. Consider a binary classification problem, let m denote the number of positive samples (class 1) and n denote the number of negative samples (class 0). Assume there are two probability density functions, Pₓ and P_{y}, such that Pₓ represents the distribution of predicted scores of positive samples and the other one P_{y} represents the predicted scores of negative samples. In the following equations, x is drawn from Pₓ, and y is drawn from P_{y}. Then the mean AUROC θ can be estimated by the Mann-Whitney statistics:

$$\hat{θ} = \frac{1}{mn} {\sum }_{i = 1}^{m} {\sum }_{j = 1}^{n} {[ {x}_{i} > {y}_{j} ]}_{} \qquad ( 2 )$$

Where m is the number of sample scores x drawn from Pₓ, n is the number of sample scores y drawn from P_{y}, [symbol] [xᵢ>yⱼ] is the characteristic function giving 1 when the condition xᵢ>yⱼ is satisfied, otherwise zero. A practical working assumption is that machine learning models seldom generate the same prediction scores for two different data points. Therefore, ties are not of great concern here in the following derivations, and hence, the situation is simplified.

$${V}_{10} ( x ) = \frac{1}{n} {\sum }_{j = 1}^{n} {[ x > {y}_{i} ]}_{} \qquad ( 3 )$$

$${V}_{01} ( y ) = \frac{1}{m} {\sum }_{i = 1}^{m} {[ {x}_{i} < y ]}_{} \qquad ( 4 )$$

$${S}_{10} = \frac{1}{m - 1} {\sum }_{i = 1}^{m} {( {V}_{10} ( {x}_{i} ) - \hat{θ} )}^{2} \qquad ( 5 )$$

$${S}_{01} = \frac{1}{n - 1} {\sum }_{j = 1}^{n} {( {V}_{01} ( {y}_{i} ) - \hat{θ} )}^{2} \qquad ( 6 )$$

Finally, the sample variance can be estimated using equation 7. An upper bound of the variance can be derived in equation 8, as both S₁₀ and S₀₁ are actually bounded by 1. To see that, θ, V₁₀(x), V₀₁(y) are all in [0,1]. What is more, s²∈[0, 1], given s∈[0, 1]. In a healthcare application of predictive models, one often faces an imbalanced dataset. It is noted that

$$\frac{1}{m} + \frac{1}{n} \approx \frac{1}{m} ,$$

$$if m << n$$

in an imbalanced setting, meaning the positive samples contribute overwhelmingly to the sample variance.

$$Var ( \hat{θ} ) = \frac{1}{m} {S}_{10} + \frac{1}{n} {S}_{01} \qquad ( 7 )$$

$$Var ( \hat{θ} ) < \frac{1}{m} + \frac{1}{n} \qquad ( 8 )$$

### Herein:

θ,θ′ is evaluation metric of the binary classifier;
□t is subscription denotes the time step;
mₜ is number of positive samples;
nₜ is number of negative samples;
zt, rt are sample mean and sample variance of the evaluation metric at current time t;
pt, t−1 are variance/covariance extrapolated based on estimation from time t−;
pt, t are current variance/covariance estimation; and
Kₜ is Kalman Gain.

Disclosed herein in a framework for estimating model performance over time. Notations and symbols are introduced in the context of binary classification problems. FIG. 6 is a diagram illustrating an example Kalman filter based framework 600 for estimating model performance over time.

Describe herein are the steps per time step iteration in Table 1 in association with FIG. 6. This algorithm is designed for AUROC, therefore zₜ is to denote the sample AUROC at time t specifically. For other model performance metrics, adjustments are needed accordingly. In the first step, sample mean AUROC zₜ is calculated using all predictions made during the current window. Sample variance rₜ is estimated by DeLong's method described above. Since an imbalanced data batch per time window is expected, if there is not enough positive samples, the upper bound of sample variance is conservatively used. Then, the previous variance estimate p_{t,t} is extrapolated to p_{t,t-1}, following DeLong's equation 7.

TABLE 1
Kalman filter steps for AUROC

| Steps | Formulas | Explanations |
|---|---|---|
| 1 | zₜ, | Estimate mean zₜ and sample variance rₜ of the |
| | If mₜ > threshold: | performance metric based on the data in the |
| | rₜ | moving window which ends at time t. Since an |
| | Else: | imbalanced data batch is expected, if there is |
| | $\frac{1}{{m}_{t}} + \frac{1}{{n}_{t}}$ | not enough positive samples, the upper bound of sample variance is conservatively used. |
| 2 | ${p}_{t , t - 1} = \frac{1}{{m}_{t}} {S}_{10 , t - 1} + \frac{1}{{n}_{t}} {S}_{01 , t - 1}$ | Extrapolate variance according to the number of positive mₜ and negative samples nₜ at |
| | | current time step. |
| 3 | ${K}_{t} = \frac{{p}_{t , t - 1}}{{p}_{t , t - 1} + {r}_{t}}$ | Calculate Kalman Gain Kₜ at time t using sample variance rₜ and the extrapolated |
| | | estimation variance p_{t,t-1} |
| 4 | θₜ = θₜ₋₁ + Kₜ(zₜ − θₜ₋₁) | Obtain the filtered value of performance metric. |
| 5 | p_{t,t} = (1 − Kₜ)p_{t,t-1} | Update variance. Equivalently, (1 − Kₜ) is |
| | | applied to S₁₀, S₀₁ at time t. p_{t,t} is used for |
| | | constructing a 95% confidence interval. |

Using a real dataset, data processing and model training steps were implemented and then the disclosed method was applied. Baseline variables and demographics were collected at the time of admission. These include initial readings of vital signs, age, gender, race, ethnicity, pregnant flag, and smoking status, number of previous hospital stays and number of previous ICU stays. No vaccine information was recorded by the Abbott Northwestern Hospital's Enterprise Data Warehouse (EDW) during the period of data collection from 2020 June to March 2023. It was determined that the cut-off admission date was Dec. 1, 2022, so that all patients included in the dataset have a known end of stay outcome. Vital signs such as systolic/diastolic blood pressure, heart rate, respiration rate, saturation of oxygen (SpO₂) and body temperature were collected multiple times a day for every patient, regardless of their Intensive Care Unit (ICU) status. Patient daily feature vectors were then derived from all readings of vitals measured in that day. Each vital sign was aggregated to mean, median, min, max, trend (slope), detrended variation and the count of measurements per day. All vital sign features (mean, median, min, max) except temperatures were log transformed following Yeo-Johnson's method. 1-SpO₂ was taken before log transformation, assuming SpO₂ was between 0 and 1. Body temperatures were standardized to z-score of a standard normal distribution. To model the temporal correlation, the first order difference of all the variables was also added in a daily feature vector. Empirically, adding higher order differences does not significantly improve performance of 5-fold cross validation on the training set.

A XGBoost classifier was trained and model parameters were tuned on the data collected until Dec. 15, 2020, and tested retrospectively on the test set collected onward until the end of 2022. Model parameters were determined using 5-fold cross validation on the training set. Learning rate was set to 0.05, L1 regularization 0.01, no L2 regularization and maximum depth to be 3. Isotonic regression was tested as a method of score calibration. However, empirically, it was found that the performance, namely AUROC, of probability calibrated classifiers were significantly worse than uncalibrated scores. Outputs of the model were then grouped by dates and ranked descendingly by their predicted scores. A daily report was generated, highlighting the patients above the threshold, the history of past predictions and the current feature importance. True positive predictions, false positive predictions and false negative predictions were randomly drawn from the test set.

The result suggests that the performance of the model remains stable through 2021 and 2022 despite the changes in class distributions, number of patients, length of stay and root causes such as evolving virus variants and improving treatment medications and guidelines.

The disclosed method utilizes a conservative upper bound of variance, which in some cases may lead to slow adaptation. However, in the specific case of AUROC of in-hospital mortality prediction models, since the performance data is already aggregated to a monthly level, this problem is mitigated in some sense. Another potential issue with long-term performance monitoring of predictive models is the problem of setting the p-value threshold and multiple comparisons. The problem of multiple comparisons against the confidence interval is a realistic concern when tracking model performance over time. The Kalman filter address this problem by shrinking the estimation of variance, as shown by step 5 in Table 1.

Besides the analysis shown herein, the filtered results were compared against the change in measurement frequency per patient per day. There were two changes of hospital operational plan during 2020 to 2022 at Abbott Northwestern hospital.

The hospital went into “crisis mode” to deal with the overwhelming number of patients and the “burnout” phenomenon of physicians and nurses. The per patient workload of nurses was reduced so that more patients could be taken care of, which means the average number of vital sign measurements per patient per day was reduced. It is noted that the model performance does not fluctuate much in response to hospital operational plan changes. It is noted that the model was trained only on 2020 data and was not retrained on new batch of data. This finding may shed lights on new possibilities of reducing the cost of healthcare while maintaining the same quality of care.

The filter of AUPRC may also be considered. Note that while sample size and class distributions are robustness factors for AUROC, they are dominant factors for AUPRC.

In summary, starting from a question which is rooted in real-world scenario, that is, how to compare performance of the same model over different time periods, through analysis, dominant and robustness factors of evaluation metrics were identified. Thus, a Kalman filter based framework is disclosed to adjust for the shift of class distributions and the change in sample size. Experiments on synthetic datasets demonstrated its ability to not only remove noises, but also to track change of performance correctly. Although the problem has healthcare contexts, the method is widely applicable and can be adapted to other performance metrics.

The following example relates to determining an optimized measurement frequency of clinical variables through variance SHAP. This example involves at least prediction analyzer 114, prediction uncertainty 116, and measurement recommendation 120 of FIG. 1 and the analyze predictions process 220 of FIG. 2C.

Disclosed herein is a view of clinical variable measurement frequency from a predictive modeling perspective, namely the measurements of clinical variables reduce uncertainty in model predictions. To achieve this goal, disclosed herein is variance SHAP with variational time series models, an application of Shapley Additive Explanation (SHAP) algorithm to attribute epistemic prediction uncertainty. The prediction variance is estimated by sampling the conditional hidden space in variational models and can be approximated deterministically by Delta's method. This approach works with variational time series models such as variational recurrent neural networks and variational transformers. Since SHAP values are additive, the variance SHAP of binary data imputation masks can be directly interpreted as the contribution to prediction variance by measurements. The disclosed method was tested on a public ICU dataset with deterioration prediction task and the relation between variance SHAP and measurement time intervals was studied.

Researchers have made enormous amount of efforts to make the deep learning black boxes transparent. Both model specific and model agnostic methods have been developed to tackle the challenge of explaining the outputs of deep learning models. Among them, the game theoretic approach of SHAP (SHapley Additive explanations) stands out and may become one of the most popular methods.

Explanations about the predictive model output alone may not be enough. To gain a holistic view of predictions, its variability needs to be understood. For future event predictions, such as patient deterioration prediction, it is also desirable to understand how soon the event will happen. Traditionally, these tasks can be done by training separate models against different targets. However, this approach is at the risk of inconsistent explanation and predictions among different models, thus it may be difficult for humans to understand. This may cause a problem when trying to translate explanation results to actions, because the causal relation between different tasks are implicit. Therefore, the only valid solution is to use one model for all tasks. Multitask learning is the most appreciated approach in this case. Alternatively, and recently, generative models are also naturally capable of performing multitasks. Disclosed herein are variational generative models to predict patient deterioration, meanwhile, desirable quantities like prediction variability, and acuity of disease (how fast the patient is deteriorating?) can be derived from the model.

Besides the purpose of seeking explanations of black-box models, the explanation of variance is crucial to the question of when and what clinical variables should be collected from patients. The solution to this question could potentially unlock methods to reduce healthcare expenses without compromising the quality of care. The problem becomes even more important in the scenario of pediatric care, where for example, frequent blood draws may do more harm than the benefit gained from test results.

Disclosed herein, the locality and additivity of SHAP values are exploited, and model predictions are expanded to prediction variability, with the help of variational models and a bit of stochastic calculus. Variational inference methods are powerful generative models which approximate the posterior distribution of assumed hidden, unobserved variables. While in variational models, the hidden states are represented by random variables, herein explicit and deterministic games of prediction variance are constructed, so that the SHAP values can be back propagated to input clinical variables. Further, additive SHAP values, propagated to carefully handcrafted features, can potentially translate to real actions. Such an example is provided herein, where it was determined that the frequency of clinical variable collections are highly correlated prediction variability.

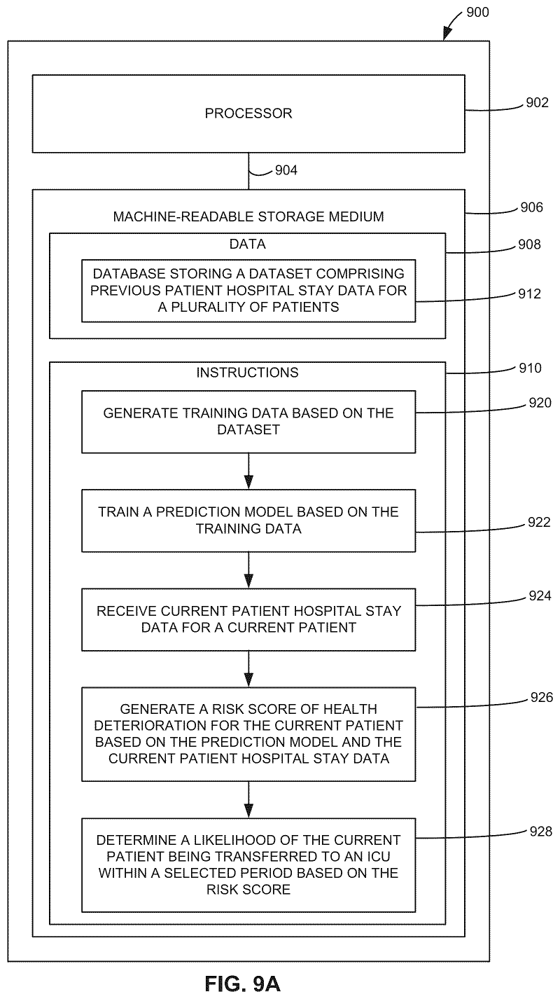

FIG. 7 is a diagram illustrating an example architecture 700 for explaining prediction variance. A variational time series model will be trained for deterioration prediction. At every time step, SHAP value explanations of predicted risk score, prediction variance are generated along with the model output.

As the name suggests, variational time series models combine variational inference with recurrent structures for time series. The term “variational time series models” refers to any time series models (i) of which its hidden states are represented by a set of parameters of some probability distributions and (ii) its hidden states are updated by some recurrence mechanism, e.g., recurrent gated unit. A wide range of models fall into this category, such as variational recurrent neural networks (VRNN), stochastic recurrent neural networks (SRNN), and variational transformers (VTrans).

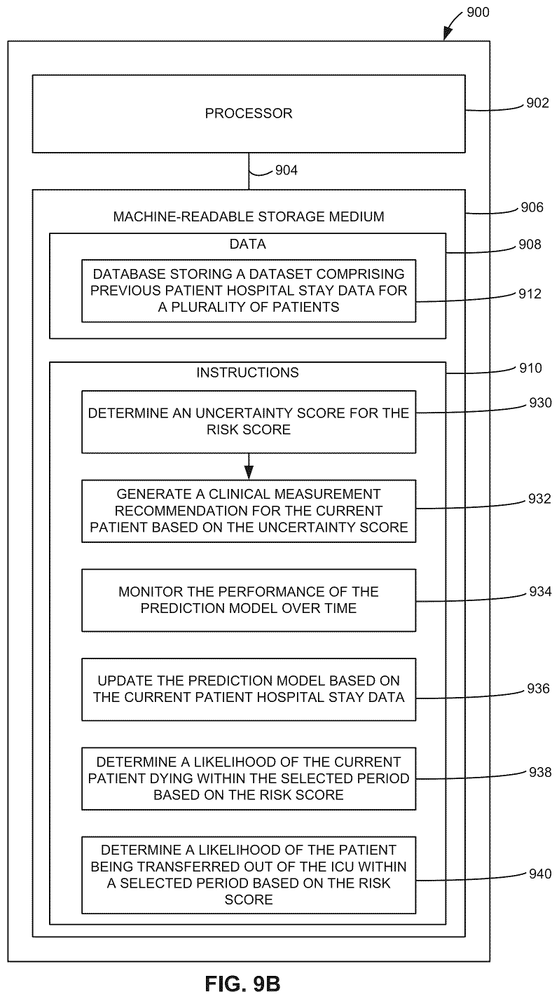

FIG. 8 shows a graphic representation of a typical variational time series model structure. Suppose the hidden space at time step t consists of several independently normally distributed variables zₜ, parameterized by mean vector μₜ and a diagonal covariance matrix Σₜ. Upon every time step forward, μₜ and Σₜ are inferred by the encoder network from current input xₜ and previous recurrent hidden state hₜ₋₁. Next, the hidden state vector is drawn from the distribution based on inferred parameters via reparameterization tricks. The task network then uses zₜ to make predictions or classifications. As for the recurrent mechanism, both zₜ and xₜ serve as the inputs to the recurrence unit. In this way, the model allows for certain degrees of stochasticity or transition between hidden states. Details of the training steps are further described below.

Formally, the input time series is denoted as x=(x₁, x₂, . . . , xₙ) where n is the length of the sequence, and subscription t is a dummy variable for time step. Each xₜ∈ [symbol] ^{d}, with d being the number of features. The deterministic hidden states from the recurrent model is marked by hₜ, while the random variable zₜ is drawn from a set of distributions parameterized by μₜ and Σₜ. θₜ is a short hand of the combination of distribution parameters μₜ and Σₜ. θ_{t,prior} stands for the parameters of prior distribution. [symbol] and yₜ denote the predicted score and the ground truth respectively. Though the main task can be of various kind such as classification, prediction or regression, CLF(⋅) is used for the main task network. Similarly, RNN(⋅) for recurrence unit, but really it can be any recurrence mechanism like Long-Short Term Memory, Gated Recurrent Unit or Transformers. The naming of other components are straightforward, ENC(·) for encoders, DEC(·) for decoders and PRIOR(·) for prior network. For parameter of distributions, μₜ, Σₜ∈ [symbol] ^{z,im}. For hidden states, hₜ∈R^{h,im}, zₜ∈ [symbol] R^{z,im}.

$${θ}_{t \_ prior} = PRIOR ( {h}_{t - 1} ) \qquad ( 9 )$$

$${θ}_{t} = ENC ( {h}_{t - 1} , {x}_{t} ) , {θ}_{t} = {μ}_{t} , {\sum }_{t}$$

$${z}_{t} = {μ}_{t} + {\sum }_{t} {ϵ}_{t} , {ϵ}_{t} \sim N ( 0 , 1 ) \qquad ( 10 )$$

$$= CLF ( {z}_{t} ) \qquad ( 11 )$$

$${h}_{t} = RNN ( {h}_{t - 1} , {x}_{t} , {z}_{t} ) \qquad ( 12 )$$

Reconstructed [symbol] is given by the decoder. Based on the result of σ-Variational Auto Encoders, it suffices to just out put [symbol] since the negative log likelihood loss is analytically determined by [symbol] itself. Therefore, there is no need for decoders to produce another set of parameters.

$$= DEC ( {h}_{t} , {z}_{t} ) \qquad ( 13 )$$

In this disclosure, variational recurrent models are trained to predict the risk of mortality of ICU patients in the next 48 hours. Clinical deterioration (also known as “clinical decompensation”) refers to the process during which the patient's condition evolves towards undesirable outcomes. Depending on the context, its meaning varies. In the emergency room, the practice of predicting deterioration is known as “triaging”. Namely, to stratify patients based on their risk of deterioration, so that patients with immediate risk will be prioritized. For patients admitted to the Intensive Care Unit (ICU), physicians are concerned with unexpected worsening of the disease and risk of mortality. For patients in general wards, clinical deterioration usually results in critical events such as transfer to the ICU or cardiopulmonary arrest. The hope is that early predictions of onsets of clinical deterioration will eventually bring benefits to all stakeholders including patients, physicians, and insurance companies. In the scope of this disclosure, the models predict ICU transfers for general ward patients and risk of mortality for ICU patients.

Although the hidden variables are modeled by parameterized distributions, the variance game (the game of attribute variance), is actually deterministic, because the variance of ŷ=cl f(z) given z˜N(μ, Σ) can be explicitly and deterministically calculated. The only thing needed to do is to wrap the original model so that the prediction variance become the wrapped model's output. Then, SHAP method can be applied. v(⋅) is used to denote the value of a game. Sampling methods are disregarded for their computational cost.

For complicated cl f functions, the problem is resolved by using Delta's method. To estimate var[f(z)], where z˜N(μ, σ), notice that

$$f ( z ) \approx f ( μ ) + ( z - μ ) {f}^{′} ( μ ) \qquad ( 14 )$$

Therefore, the variance can be estimated by:

$$var [ f [ z ] ] = var [ f ( z - μ ) ] \cdot {[ {f}^{′} ( μ ) ]}^{2} = {σ}^{2} \cdot {[ {f}^{′} ( μ ) ]}^{2} \qquad ( 15 )$$

Medical Information Mart for Intensive Care, or MIMIC-IV in short, is a large, open-source, deidentified database of hospitalized patients. It contains clinical notes, ECG data, time series of vital signs, laboratory test results and assessment scores. In this disclosure, MIMIC4 data for ICU patients is used which contains about 60,000 ICU stays after data cleaning. The data is aggregated to hourly level time series of varying lengths. The median length of stay is 61 hours, while the mortality rate is 7.8%. Variational recurrent models are trained to predict the risk of mortality in the next 48 hours. All variables are normalized and sanity checked (for example, heart rate cannot be negative, saturation of oxygen must be within 0 to 100, all temperatures share the same units, etc.). The process left 176 time series variables. Ten of them were picked after extensive feature selections. In addition, a mask is associated with each variable indicating whether the value is missing and imputed or actually measured. Log base 24 is also applied to time intervals between measurements. Therefore, there is a total of 30 features per time step. The dataset is split to training, validation and test set, controlling both mortality rate and length of stay.

To model the frequency of measurements, features that represents the log time interval from the last valid measurements are simply handcrafted. In this way, by checking the variance SHAP contribution of these variables, questions are answered: how frequently should a specific clinical variable be measured? Due to the size of the data, 8,000 patients were randomly sampled from training set as background. Noticeably, unexpected patterns were observed for systolic blood pressure measurements. The longer the time interval between two blood pressure measurements, the lower prediction variance there will be.

For measured variables which contribute (either positively or negatively) little to both predicted risk score and prediction variance, these measurements are defined as potentially “avoidable measurements”. For variables of which its missingness contribute significantly to prediction variance, these measurements are marked as potentially “should-have measurements”.

It is acknowledged that there are works pointing to the problems of SHAP values. For example, some works point out that SHAP does not work well with time series models. However, SHAP attributions were not observed as chaotic using the method disclosed herein. Therefore, the normalization technique of temporal saliency mapping (TSR) was not applied herein.

In some examples, since the Delta's method is an approximation of prediction variance, to gain more accurate estimations, it may be desirable to further expand the Taylor series to include the second order derivatives.

The disclosed method may also be applicable to determining the reason behind abnormal patterns. Since SHAP value measure the difference between local feature contribution to expected output, looking into the absolute value of variance contribution may also be helpful in clinical settings. Another potential application is to search for potentially avoidable order of lab tests without compromising the quality of care. Thus, the cost can be reduced. This would be useful especially for pediatric care, where frequent blood draws may bring more harm than benefits.

Training may proceed as follows. Since the integral of joint probability p (x, z) over the entire hidden space,

$${\int }_{z} p ( x , {z}^{′} ) {dz}^{′} \qquad ( 16 )$$

is intractable, variational are trained on evidence lower bound (ELBo), given by:

$$ELBo = {lnp}_{θ} ( x \vert z , h ) - KLdivergence [ {p}_{θ} ( z \vert x , h ) ‖ {p}_{{θ}_{prior}} ( h ) ] \qquad ( 17 )$$

Maximizing ElBo is equivalent to minimizing L_{kld}+L_{NLL}, defined below. Subscription t applies to all variables above, hence is omitted.

The training of variational time series models shares similarities with variational auto encoders (VAE): a Kullback-Leibler divergence between posterior θₜ and prior θ_{t,prior} and a regularization loss on θₜ. The approach in o-Variational Auto Encoders is adopted such that:

$${ℒ}_{{total}^{kld}} = KLdivergence [ {p}_{θ t} ( {z}_{t} \vert {x}_{t} , {h}_{t} ) ‖ {p}_{θ t , prior} ( {h}_{t} ) ] \qquad ( 18 )$$

$${p}_{θ t} ( {z}_{t} \vert {x}_{t} , {h}_{t} ) \sim N ( {μ}_{t} , {\sum }_{t} )$$

$${p}_{θ t , prior} ( {z}_{t} \vert {h}_{t} ) ∼ N ( {μ}_{t , prior} , {\sum }_{t , prior} )$$

$${ℒ}_{σ - NLL} = NLL ( , {x}_{t} , log ( σ ) \qquad ( 19 )$$

$$log ( σ ) = log ( \sqrt{{( - {x}_{t} )}^{2}} )$$

Notice that there are different ways of choosing a prior, depending on the specific problem settings. These two losses are essential components for variational inference. Additionally, there is the prediction or classification loss from the main task network and the reconstruction loss to train recurrent networks. MSE denotes the mean squared error.

$${ℒ}_{clf} = Cross - Entropy ( , {y}_{t} ) \qquad ( 20 )$$

$${ℒ}_{recon} = MSE ( , {x}_{t} )$$

Therefore the total loss per time step is given by the following equation.

$${ℒ}_{total} = {ℒ}_{kld} + {ℒ}_{σ - NLL} + {ℒ}_{clf} + {ℒ}_{recon} + {λℒ}_{reg} \qquad ( 21 )$$

Depending on whether a complete sequence can be seen at the time of inference, the loss may be average over all the time step. Also, regularization terms [symbol] _{reg} may be appended to stabilize and accelerate training. λ controls the strength of regularization.

Ten features with the least missingness rate may be selected. They are:
Diastolic blood pressure
Systolic blood pressure
Arterial Mean blood pressure
Fraction of inspired O₂
Glucose, blood
Heart rate
PH value, blood
Respiration rate
Saturation of Oxygen
Body temperature
   VRNN models trained on 10, 30, 50 and all time series features were compared. No significant performance gap was found when trained on the same random split with various number of features.

Since the advent of SHAP, it has been extensively applied to many areas, such as geology, finance, and healthcare. Recently, SHAP values have been applied to gaussian process. The variance of SHAP value (which is necessary for inferences of gaussian process) is not the SHAP value of the variance game (the focus in this disclosure). SHAP has also been combined with variational auto encoders (VAE) to explain feature contributions. The beauty of SHAP values is appreciated since it is model agnostic and flexible to most type of model outputs. To take a step further, if there exists a function calculating the variance of the prediction, SHAP methods can be applied as well.

It is also noted that there are plenty of approaches other than SHAP values, especially for time series data. However, as described above, it appears that SHAP is the best fit for this disclosure. Besides, studies have shown several drawbacks of SHAP, noticeably with entangled time series features. But as described below, with the power of variational time series models, this defect is mitigated by the inference of independent hidden state variables and thus not a major concern.

The predicted probability score by machine learning models contains two sources of uncertainty, aleatoric and epistemic uncertainty. While the latter one comes from the uncertainty about model parameters, aleatoric originates from data and unobserved factors. With aleatoric uncertainty and variance of prediction scores in focus, Bayesian methods and variational inference methods become natural choices for estimating prediction variance, with the assumption that hidden state random variables approximate the posterior distribution of inputs. This fundamental assumption should hold true for every variational generative model, for models to be fully effective. In this disclosure the focus is on how to explain the contributions of input clinical features to prediction variance.

In an application area like healthcare, explainability and interpretability are crucial features to build trustworthy machine learning and AI applications. SHAP values is the dominant approach in recent studies about explainable healthcare machine learning models. While few studies have focused on explaining deep learning models, fewer studies have focused on explaining time series in healthcare. The most related work relates to use of variational auto encoders to study multi-omics data for cancer diagnostic.

The disclosed method was verified by training a unidirectional variational recurrent neural network on MNIST. The training took 10 epochs and achieved an accuracy of 98%. The following were compared: (a) SHAP values of predicted class probabilities, (b) Variance of the prediction SHAP, (c) (proposed) SHAP value of prediction variance attribution. The first thing noticed was that as expected, the attribution of variance and prediction do not coincide. Namely the model can be very confident on the prediction with a high predicted probability score (high score with low variance), or the model can be sure that the input instance is not one of the target class (low score with low variance). Vice versa, there could be the case where high score and high variance coexist. Additionally, SHAP attributions as chaotic were not observed. Therefore, the normalization technique of temporal saliency mapping (TSR) was not applied.

FIGS. 9A and 9B are block diagrams illustrating an example processing system 900 for determining a risk score. In some examples, processing system 900 is used to implement system 100 of FIG. 1. Processing system 900 includes a processor 902 and a machine-readable storage medium 906 (e.g., memory). In some examples, processor 902 may implement at least a portion of data processor 104, model trainer 110, prediction analyzer 114, and performance monitor 124 of FIG. 1. In some examples, machine readable storage medium 906 may store at least a portion of raw data 102, historical data 106, daily increment 108, model 112, and outputs 115 of FIG. 1.

Processor 902 is communicatively coupled to machine-readable storage medium 906 through the communication path 904. Although the following description refers to a single processor and a single machine-readable storage medium, the description may also apply to a system with multiple processors and multiple machine-readable storage mediums. In such examples, the instructions may be distributed (e.g., stored) across multiple machine-readable storage mediums and the instructions may be distributed (e.g., executed by) across multiple processors.

Processor 902 includes one (i.e., a single) central processing unit (CPU) or microprocessor or more than one (i.e., multiple) CPU or microprocessor, and/or other suitable hardware devices for accessing data 908 and for retrieval and execution of instructions 910 stored in machine-readable storage medium 906. Processor 902 may access a database 912 storing a dataset comprising previous patient hospital stay data for a plurality of patients and fetch, decode, and execute instructions 920-940 to operate a prediction modeling system, such as system 100 of FIG. 1. In some examples, the previous patient hospital stay data comprises clinical features, vital signs, demographics, and intensive care unit (ICU) status for the plurality of patients (e.g., EHR data). In some examples, the dataset comprises previous patient hospital stay data for ICU patents (e.g., MIMIC3 or MIMIC4).

As shown in FIG. 9A, processor 902 may fetch, decode, and execute instructions 920 to generate training data based on the dataset. Processor 902 may fetch, decode, and execute instructions 922 to train a prediction model (e.g., 112 of FIG. 1, 308 of FIG. 3) based on the training data. Processor 902 may fetch, decode, and execute instructions 924 to receive current patient hospital stay data for a current patient. In some examples, the current patient hospital stay data comprises clinical features, vital signs, and/or demographics for the current patient. In some examples, the current patient has a disease different from the plurality of patients in the dataset. Processor 902 may fetch, decode, and execute instructions 926 to generate a risk score (e.g., 118 of FIG. 1) of health deterioration for the current patient based on the prediction model and the current patient hospital stay data. In some examples, the processor is configured to execute the instructions to generate the risk score based on feature importance of the current patient hospital stay data. Processor 902 may fetch, decode, and execute instructions 928 to determine a likelihood of the current patient being transferred to an ICU within a selected period (e.g., within a range between 24 to 96 hours) based on the risk score.

As shown in FIG. 9B, processor 902 may fetch, decode, and execute further instructions 930 to determine an uncertainty score (e.g., 116 of FIG. 1) for the risk score. Processor 902 may fetch, decode, and execute further instructions 932 to generate a clinical measurement recommendation (e.g., 120 of FIG. 1) for the current patient based on the uncertainty score. Processor 902 may fetch, decode, and execute further instructions 934 to monitor the performance of the prediction model over time (e.g., via 124 of FIG. 1 or 400 of FIG. 4). Processor 902 may fetch, decode, and execute further instructions 936 to update the prediction model (e.g., using 108 and 122 of FIG. 1) based on the current patient hospital stay data. Processor 902 may fetch, decode, and execute further instructions 938 to determine a likelihood of the current patient dying within the selected period based on the risk score. Processor 902 may fetch, decode, and execute further instructions 940 to determine a likelihood of the patient being transferred out of the ICU within a selected period based on the risk score.

As an alternative or in addition to retrieving and executing instructions, processor 902 may include one (i.e., a single) electronic circuit or more than one (i.e., multiple) electronic circuits comprising a number of electronic components for performing the functionality of one of the instructions or more than one of the instructions 910 in machine-readable storage medium 906. With respect to the executable instruction representations (e.g., boxes) described and illustrated herein, it should be understood that part or all of the executable instructions and/or electronic circuits included within one box may, in alternate examples, be included in a different box illustrated in the figures or in a different box not shown.

Machine-readable storage medium 906 is a non-transitory storage medium and may be any suitable electronic, magnetic, optical, or other physical storage device that stores executable instructions. Thus, machine-readable storage medium 906 may be, for example, a random access memory (RAM), an electrically-erasable programmable read-only memory (EEPROM), a storage drive, an optical disc, and the like. Machine-readable storage medium 906 may be disposed within system 900, as illustrated in FIGS. 9A and 9B. In this case, the executable instructions may be installed on system 900. Alternatively, machine-readable storage medium 906 may be a portable, external, or remote storage medium that allows system 900 to download the instructions from the portable/external/remote storage medium. In this case, the executable instructions may be part of an installation package.

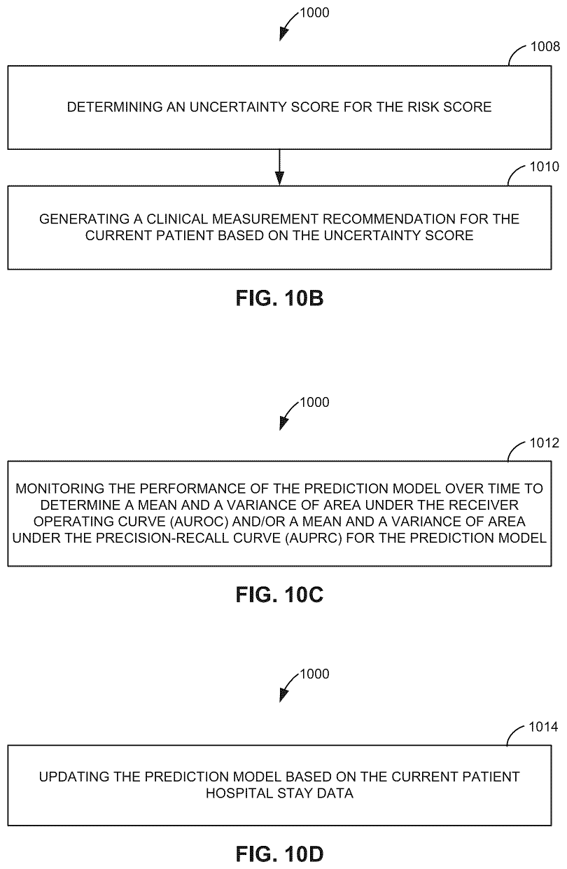

FIGS. 10A-10C are flow diagrams illustrating an example method 1000 for determining a risk score. In some examples, method 1000 may be implemented by system 100 of FIG. 1 or system 900 of FIGS. 9A and 9B. As illustrated in FIG. 10A at 1002, method 1000 may include generating a prediction model (e.g., 112 of FIG. 1 or 308 of FIG. 3) based on a dataset (e.g., 102 of FIG. 1) comprising previous patient hospital stay data including clinical features, vital signs, demographics, and intensive care unit (ICU) status for a plurality of patients. In some examples, the previous patient hospital stay data represents a cohort different from a cohort of the current patient, and generating the prediction model comprises generating the prediction model via transfer learning (e.g., via 300 of FIG. 3). At 1004, method 1000 may include determining a risk score (e.g., 118 of FIG. 1) of health deterioration of a current patient based on the prediction model and current patient hospital stay data to determine a likelihood of the current patient being transferred to an ICU within a selected period. At 1006, method 1000 may include adjusting treatment of the current patient and/or preparing the ICU to receive the current patient in response to the likelihood of the current patient being transferred to the ICU within the selected period exceeding a threshold.

As illustrated in FIG. 10B at 1008, method 1000 may further include determining an uncertainty score (e.g., 116 of FIG. 1) for the risk score. At 1010, method 1000 may further include generating a clinical measurement recommendation (e.g., 120 of FIG. 1) for the current patient based on the uncertainty score. As illustrated in FIG. 10C at 1012, method 1000 may further include monitoring the performance of the prediction model over time (e.g., via 124 of FIG. 1 or 400 of FIG. 4) to determine a mean and a variance of Area Under the Receiver Operating Curve (AUROC) and/or a mean and a variance of Area Under the Precision-Recall Curve (AUPRC) for the prediction model. As illustrated in FIG. 10D at 1014, method 1000 may further include updating the prediction model (e.g., using 108 and 122 of FIG. 1) based on the current patient hospital stay data.

It is to be understood that the features of the various exemplary embodiments described herein may be combined with each other, unless specifically noted otherwise.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

What is claimed is:

1. A system comprising:
a database storing a dataset comprising previous patient hospital stay data for a plurality of patients;
a memory storing instructions; and
a processor communicatively coupled to the memory and the database, the processor configured to execute the instructions to:
generate training data based on the dataset;
train a prediction model based on the training data;
receive current patient hospital stay data for a current patient;
generate a risk score of health deterioration for the current patient based on the prediction model and the current patient hospital stay data; and
determine a likelihood of the current patient being transferred to an ICU within a selected period based on the risk score.

2. The system of claim 1, wherein the previous patient hospital stay data comprises clinical features, vital signs, demographics, and intensive care unit (ICU) status for the plurality of patients.

3. The system of claim 1, wherein the current patient hospital stay data comprises clinical features, vital signs, and/or demographics for the current patient.

4. The system of claim 1, wherein the processor is configured to execute the instructions to determine an uncertainty score for the risk score.

5. The system of claim 4, wherein the processor is configured to execute the instructions to generate a clinical measurement recommendation for the current patient based on the uncertainty score.

6. The system of claim 1, wherein the processor is configured to execute the instructions to monitor the performance of the prediction model over time.

7. The system of claim 1, wherein the processor is configured to execute the instructions to generate the risk score based on feature importance of the current patient hospital stay data.

8. The system of claim 1, wherein the processor is configured to execute the instructions to update the prediction model based on the current patient hospital stay data.

9. The system of claim 1, wherein the processor is configured to execute the instructions to determine a likelihood of the current patient dying within the selected period based on the risk score.

10. The system of claim 1, wherein the processor is configured to execute the instructions to determine a likelihood of the patient being transferred out of the ICU within a selected period based on the risk score.

11. The system of claim 1, wherein the selected period is within a range between 24 hours and 96 hours.

12. The system of claim 1, wherein the dataset comprises previous patient hospital stay data for ICU patents; and
wherein the current patient has a disease different from the plurality of patients in the dataset.

13. A system comprising:
a data processor configured to generate training data for a predication model based on previous patient hospital stay data for a plurality of patients and to generate a risk score for health deterioration for a current patient based on the prediction model and current patient hospital stay data;
a prediction model trainer configured to train the prediction model based on the training data;
a prediction analyzer configured to generate an uncertainty score for the risk score and to generate a clinical measurement recommendation for the current patient based on the uncertainty score; and
a prediction model performance monitor configured to monitor a performance of the prediction model over time.

14. The system of claim 13, wherein the predication model performance monitor comprises a Kalman filter based framework.

15. The system of claim 13, wherein the prediction model comprises an extreme Gradient Boosting (XGBoost) prediction model.

16. A method comprising:
generating a prediction model based on a dataset comprising previous patient hospital stay data including clinical features, vital signs, demographics, and intensive care unit (ICU) status for a plurality of patients;
determining a risk score of health deterioration of a current patient based on the prediction model and current patient hospital stay data to determine a likelihood of the current patient being transferred to an ICU within a selected period; and
adjusting treatment of the current patient and/or preparing the ICU to receive the current patient in response to the likelihood of the current patient being transferred to the ICU within the selected period exceeding a threshold.

17. The method of claim 16, further comprising:
determining an uncertainty score for the risk score; and
generating a clinical measurement recommendation for the current patient based on the uncertainty score.

18. The method of claim 16, further comprising:
monitoring the performance of the prediction model over time to determine a mean and a variance of Area Under the Receiver Operating Curve (AUROC) and/or a mean and a variance of Area Under the Precision-Recall Curve (AUPRC) for the prediction model.

19. The method of claim 16, further comprising:
updating the prediction model based on the current patient hospital stay data.

20. The method of claim 16, wherein the previous patient hospital stay data represents a cohort different from a cohort of the current patient, and
wherein generating the prediction model comprises generating the prediction model via transfer learning.
